(19) United States
(12) Patent Application Publication
(10) Pub. No.: US 2012/0135057 A1
(43) Pub. Date: May 31, 2012

(54) ANTIPERSPIRANT COMPOSITIONS AND PRODUCTS HAVING A DISPERSED COOLING SENSATION AGENT AND METHODS FOR MAKING THE SAME

(75) Inventors: Travis T. Yarlagadda, Phoenix, AZ (US); Thomas Doering, Scottsdale, AZ (US)

(73) Assignee: The Dial Corporation, Scottsdale, AZ (US)

(21) Appl. No.: 12/956,166

(22) Filed: Nov. 30, 2010

(51) Int. Cl. A61K 8/02; B29C 39/02; A61Q 15/00; A61K 8/25; A61K 8/97

(52) U.S. Cl. 424/401; 424/65; 424/47; 264/240

(57) ABSTRACT

A personal care product comprises an antiperspirant product that is housed within a container. The antiperspirant product comprises a dispersed cooling sensation agent that is adsorbed onto surfaces of silica particles that are dispersed throughout the antiperspirant product, absorbed into pores of the silica particles or a combination thereof.

### FIELD OF THE INVENTION

The present invention generally relates to personal care antiperspirant compositions, products, and methods for making the same, and more particularly relates to antiperspirant compositions that exhibit antiperspirant efficacy and cooling sensation effects, antiperspirant products comprising such antiperspirant compositions, and methods for manufacturing such antiperspirant compositions and products.

### BACKGROUND OF THE INVENTION

Antiperspirant and deodorant compositions are well known personal care products used to prevent or eliminate perspiration and body odor caused by perspiration. The compositions come in a variety of forms and may be formulated, for example, into aerosols, pumps, sprays, liquids, roll-ons, lotions, creams, and sticks (both hard and soft), etc.

There are various types of stick antiperspirant compositions that are desirable by a large majority of the population because of their ease of application and the presence of active antiperspirant compounds, e.g. antiperspirant salts, that prevent or block the secretion of perspiration and its accompanying odors. In one type, an antiperspirant salt is suspended in an anhydrous vehicle often including a solid water-insoluble wax. In a second type, an antiperspirant salt is dissolved in a liquid vehicle such as propylene glycol and gelled with a gelling agent such as dibenzylidene sorbitol. A third type includes an emulsion of an aqueous phase containing the antiperspirant salt and an oil phase containing, for example, a volatile silicone, fragrances, gellants, and other additives.

Stick antiperspirant products include an antiperspirant composition within a container. During use of the product, the top of the container is removed and the application surface of the composition is contacted with the skin, such as the underarm, by swiping or rubbing the stick across the skin. Sometimes the product also includes an undercap, or factory seal, covering the application surface that is removed prior to first use. The container generally also includes some mechanism for moving the composition upwards through the container to continue to provide an exposed application surface.

Commercial markets for antiperspirant and deodorant products are highly competitive, with consumers wanting increased antiperspirant efficacy and refreshing effects from these products. One problem for antiperspirant and deodorant manufacturers is that the ingredients and/or additives used in the products for antiperspirant efficacy and refreshing effects may be quite volatile and readily evaporate during production and subsequent use thereafter. Consequently, these products may have limited shelf life and become less effective and/or less refreshing over time. Another problem is that such antiperspirant or deodorant compositions may not provide efficacy and refreshing effects when the user needs it most throughout the day.

Accordingly, it is desirable to provide antiperspirant products that exhibit strong antiperspirant efficacy and refreshing effects, and which have robust compositions for manufacturing, good shelf life and/or are more responsive to the user when needed most. In addition, it is desirable to provide methods for manufacturing such antiperspirant products. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Antiperspirant products and methods for manufacturing antiperspirant products are provided herein. In accordance with an exemplary embodiment, a personal care product is provided. The personal care product comprises a container. An antiperspirant product is housed within the container and comprises a dispersed cooling sensation agent that is adsorbed onto surfaces of silica particles that are dispersed throughout the antiperspirant product, absorbed into pores of the silica particles or a combination thereof.

In accordance with another exemplary embodiment, a method for manufacturing an antiperspirant product is provided. The method comprises depositing the antiperspirant product into a mold. The antiperspirant product comprises a dispersed cooling sensation agent that is adsorbed onto surfaces of silica particles that are dispersed throughout the antiperspirant product, absorbed into pores of the silica particles or a combination thereof. The antiperspirant product is allowed to solidify.

### BRIEF DESCRIPTION OF THE DRAWINGS

The present invention will hereinafter be described in conjunction with the following drawing figures, wherein like numerals denote like elements, and

FIG. 1 is a perspective view of a personal care antiperspirant product in accordance with an exemplary embodiment;

FIG. 2 is a plan view of the antiperspirant product of FIG. 1;

FIG. 3 is an exploded perspective view of the antiperspirant product of FIG. 1 in accordance with an exemplary embodiment;

FIG. 4 is a flowchart of an example of a process for manufacturing an antiperspirant composition and product in accordance with the present invention; and

FIG. 5 is a side cutaway view of a personal care antiperspirant product in accordance with another exemplary embodiment.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to personal care antiperspirant products that exhibit antiperspirant efficacy and provide a cooling sensation effect to a user upon perspiration. The antiperspirant product comprises an active antiperspirant ingredient or ingredients and a cooling sensation agent that is adsorbed onto surfaces and/or absorbed into pores of a plurality of silica particles that are dispersed throughout the product. By capturing and/or carrying the cooling sensation agent on the silica particles, the inventors have unexpectedly found that the cooling sensation agent does not readily evaporate including during manufacturing of the product, thereby minimizing evaporative loss of the cooling sensation agent from the antiperspirant composition. In one exemplary embodiment, the silica particles are porous and hydrophilic such that when the antiperspirant product is applied to an underarm and the user perspires, the perspiration is readily absorbed by the silica particles, displacing or driving out the cooling sensation agent. As such, the cooling sensation agent intermingles with the perspiration and is absorbed into the skin, producing a cooling sensation effect that is perceived by the user. The inventors have also found that by capturing and/or carrying the cooling sensation agent on the silica particles that are effective to release the cooling sensation agent when contacted with perspiration, the antiperspirant product has an excellent shelf-life and provides the user a cooling sensation effect throughout the day when it is most needed, e.g., when the user perspires.

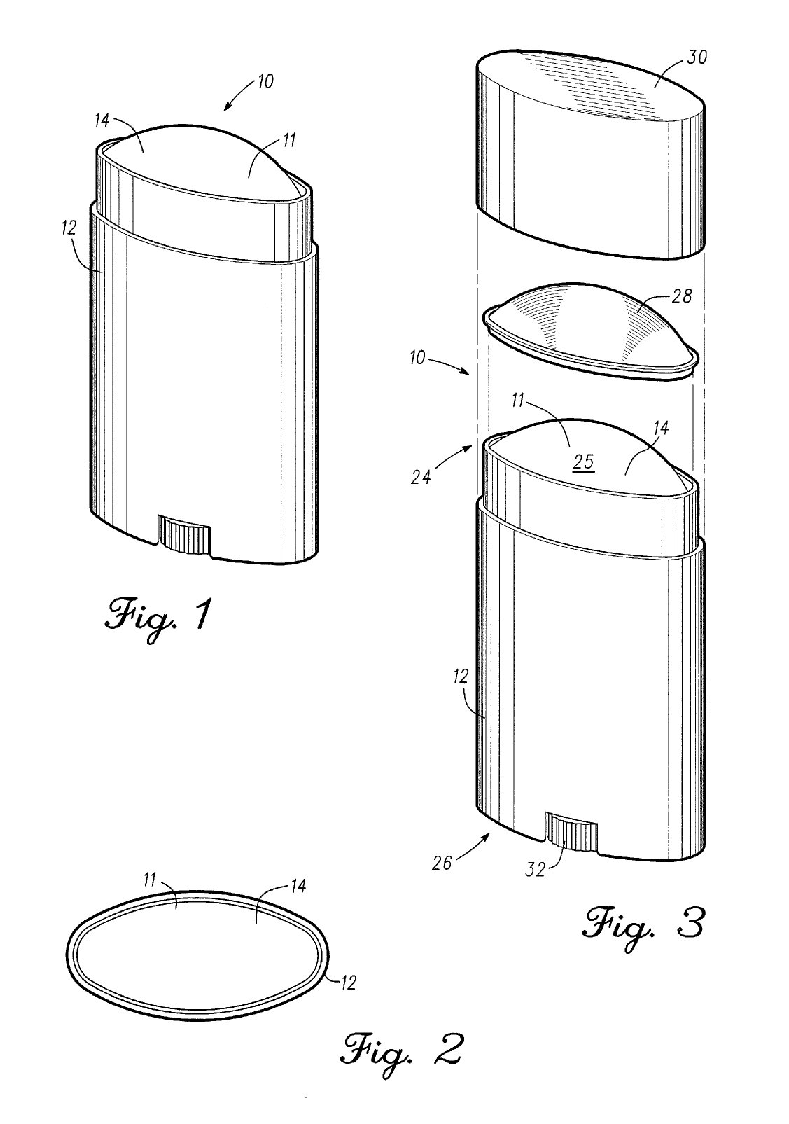

Referring to FIGS. 1 and 2, a personal care product 10 in accordance with an exemplary embodiment comprises an antiperspirant product 11. The antiperspirant product 11 is preferably a solid wax formulation, however, other suitable antiperspirant formulations may be used, such as, for example, an antiperspirant aerosol formulation as depicted in FIG. 5. The antiperspirant product 11 has an application surface 14 that is substantially dome-shaped and that is configured to be applied to skin, such as, for example, an underarm. The personal care product 10 may also comprise a container or dispenser 12 for dispensing the antiperspirant product 11 to the skin.

In one exemplary embodiment, the antiperspirant product 11 contains a cooling sensation agent that is adsorbed onto surfaces and/or absorbed into pores of silica particles that are in the form of a solid powder dispersed throughout the antiperspirant product 11. As such, the cooling sensation agent is effectively dispersed throughout the antiperspirant product 11. By keeping the cooling sensation agent captured and/or affixed to the silica particles, the inventors have found that the cooling sensation efficacy of the antiperspirant product 11 is better maintained.

Non-limiting examples of cooling sensation agents are menthol, vanillyl butyl ether, peppermint oil, methane carboxamide ethyl pyridine, menthoxypropanediol, menthanediol, cyanomethylphenyl methane carboxamide, camphor, ethyl menthane carboxamide, menthyl diisopropyl propionamide, menthyl lactate, 4-(butoxymenthyl)-2-methoxy-phenol, 3-[[5-methyl-2-(1-methyl)cyclohexyl]oxy]-1,2-propanediol, isopulegol, or a mixture thereof. In one example, the cooling sensation agent is either Winsense® Extra 400 or Winsense® Extra 500, which each comprise a mixture of ethyl menthane carboxamide, menthyl diisopropyl propionamide and menthyl lactate in various proportions. Winsense® Extra 400 and Winsense® Extra 500 are manufactured by LyondellBasell of Rotterdam, the Netherlands. Other suitable cooling sensation agents known to those skilled in the art may also be used. The antiperspirant product 11 preferably contains an amount of the cooling sensation agent of from about 0.2 to about 2.5 weight percent (wt. %). The term “about” as used herein means within typical manufacturing tolerances. In another exemplary embodiment, the antiperspirant product 11 is an aerosol formulation (see FIG. 5) and preferably contains an amount of cooling sensation agent of from about 0.1 to about 2 wt. %.

The silica particles are preferably porous and hydrophilic. In an exemplary embodiment, the silica particles are hydrophilic forms of precipitated silica having a BET surface area (the acronym “BET” originating from the first initials of the family names of the developers of the surface area measurement method, e.g., Stephen Brunauer, Paul Emmett and Edward Teller) of from about 50 to about 1000 m²/g, preferably from about 100 to about 700 m²/g, and most preferably from about 150 to about 600 m²/g; a particle size d₅₀, determined by laser diffraction, from about 2 to about 130 μm, preferably from about 3 to about 20 μm; and a DOA (dioctyl acetate) absorption from about 150 to about 400 g DBP/100 g silica, preferably from about 200 to about 350 g DBP/100 g silica. One such type of silica is Sipernat® 22S, which is manufactured and sold by Evonik Industries of Mobile, Ala. and is comprised of fine silica particles with high absorption capacity for liquids, especially water. The cooling sensation agent is adsorbed onto, absorbed into and effectively captured by the porous hydrophilic silica particles. When the porous hydrophilic silica particles are exposed to an amount of moisture, e.g. perspiration, the moisture is readily taken up by the porous hydrophilic silica particles displacing and releasing (e.g. eluting) the cooling sensation agent from the particles. Preferably, the antiperspirant product 11 comprises the silica particles in an amount of from about 0.5 to about 4 wt. %. In another exemplary embodiment, the antiperspirant product 11 is an aerosol formulation (see FIG. 5) and preferably comprises the silica particles in an amount of from about 0.1 to about 4 wt. %. Other suitable silica particles or hydrophilic carriers known to those skilled in the art may also be used to disperse the cooling sensation agent throughout the antiperspirant product 11.

Additionally, the antiperspirant product 11 may contain a relatively low amount of an unbound cooling sensation agent that is not captured by or carried on the silica particles. The unbound cooling sensation agent preferably provides the cooling sensation effect when the antiperspirant product is initially applied to the user and for some period throughout the day thereafter.

The antiperspirant product 11 may comprise an anhydrous, hydrophobic vehicle, which includes a volatile silicone and/or high melting component, and an active antiperspirant compound suspended in the anhydrous, hydrophobic vehicle. The active antiperspirant compound is preferably in a perspiration-reducing effective amount.

The active antiperspirant compounds contain at least one active ingredient, typically metal salts, that are thought to reduce perspiration by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x(H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐx(H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the antiperspirant compound is aluminum zirconium pentachlorohydrex. In another embodiment, the antiperspirant product 11 comprises an active antiperspirant compound present in the amount of 0 to about 25 wt. % (USP). As used herein, weight percent (USP) or wt. % (USP) of an antiperspirant salt is calculated as anhydrous weight percent in accordance with the U.S.P. method, as is known in the art. This calculation excludes any bound water and glycerin. In a more preferred embodiment, the antiperspirant compound comprises aluminum zirconium pentachlorohydrex at an active level of about 18 to about 24 wt. % (USP).

The high melting components may include any material suitable for use in an antiperspirant stick that melts at a temperature of about 70° C. or higher. Typical of such materials are the high melting point waxes. These include beeswax, spermaceti, carnauba, bayberry, candelilla, montan, ozokerite, ceresin, paraffin waxes, semi-microcrystalline and microcrystalline waxes, hydrogenated jojoba oil, and hydrogenated castor oil (castor wax). The preferred wax is hydrogenated castor oil. Other suitable high melting components include various types of high melting gelling agents such as polyethylene-vinyl acetate copolymers, polyethylene homopolymers, 12-hydroxystearic acid, and substituted and unsubstituted dibenzylidene alditols. Typically, the high melting components comprise about 1 to about 25 wt. %, preferably about 2 to about 15 wt. %, of the composition. Volatile silicones include cyclomethicones and dimethicones, discussed above.

Other components may include, for example, non-volatile silicones, polyhydric alcohols having 3-6 carbon atoms and 2-6 hydroxy groups, fatty alcohols having from 12 to 24 carbon atoms, fatty alcohol esters, fatty acid esters, fatty amides, non-volatile paraffinic hydrocarbons, polyethylene glycols, polypropylene glycols, polyethylene and/or polypropylene glycol ethers of C₄-C₂₀ alcohols, polyethylene and/or polypropylene glycol esters of fatty acids, and mixtures thereof. The term “fatty” is intended to include hydrocarbon chains of about 8 to 30 carbon atoms, preferably about 12 to 18 carbon atoms.

Non-volatile silicones include polyalkylsiloxanes, polyalkylaryl siloxanes, and polyethersiloxanes with viscosities of about 5 to about 100,000 centistokes at 25° C., polymethylphenylsiloxanes with viscosities of about 15 to about 65 centistokes, and polyoxyalkylene ether dimethylsiloxane copolymers with viscosities of about 1200 to about 1500 centistokes.

Useful polyhydric alcohols include propylene glycol, butylenes glycol, dipropylene glycol and hexylene glycol. Fatty alcohols include stearyl alcohol, cetyl alcohol, myristyl alcohol, oleyl alcohol, and lauryl alcohol. Fatty alcohol esters include C₁₂₋₁₅ alcohols benzoate, myristyl lactate, cetyl acetate, and myristyl octanoate. Fatty acid esters include isopropyl palmitate, myristyl myristate, and glyceryl monostearate. Fatty amides include stearamide MEA, stearamide MEA-stearate, lauramide DEA, and myristamide MIPA.

Non-volatile paraffinic hydrocarbons include mineral oils and branched chain hydrocarbons with about 16 to 68, preferably about 20 to 40, carbon atoms. Suitable polyethylene glycols and polypropylene glycols will typically have molecular weights of about 500 to 6000, such as PEG-10, PEG-40, PEG-150 and PPG-20, often added as rheology modifiers to alter product appearance or sensory attributes.

Polyethylene and/or polypropylene glycol ethers or C₄-C₂₀ alcohols include PPG-10 butanediol, PPG-14 butyl ether, PPG-5-buteth-7, PPG-3-isostearth-9, PPG-3-myreth-3, oleth-10, and steareth-20. Polyethylene and/or polypropylene glycol esters of fatty acids include PEG-8 distearate, PEG-10 dioleate, and PPG-26 oleate. These are generally added to give emollient properties.

The antiperspirant product 11 contemplated herein also may comprise additives, such as those used in conventional antiperspirants. For example, in addition to, or instead of, antiperspirant efficacy, the antiperspirant product 11 may comprise additives that cause the antiperspirant product 11 to exhibit long-lasting fragrance, odor protection, bacteria control, and/or another desired purpose and/or function. These additives include, but are not limited to, fragrances, including encapsulated fragrances, dyes, pigments, preservatives, antioxidants, moisturizers, and the like. These optional ingredients can be included in the antiperspirant product 11 in an amount of 0 to about 20 wt. %.

The above list of materials is by way of example only and is not intended to be a comprehensive list of all potential components of the antiperspirant products contemplated herein. Other high and low melting waxes, volatile and non-volatile compounds and other suitable components are readily identifiable to those skilled in the art. Of course, other ingredients such as colloidal silica, fumed silica, particulate polyolefins, talcum materials, colorants and preservatives may also be included as desired. For example, the composition may include up to about 10% fragrance or about 2% colorant by weight.

As noted above, in addition to, or instead of, an active antiperspirant compound, the antiperspirant product 11 may comprise a component or components that cause it to exhibit or impart a desired function or purpose in addition to, or instead of, antiperspirant efficacy. For example, the antiperspirant product 11 may comprise deodorant active ingredients. A suitable deodorant active ingredient is any agent that inhibits, suppresses, masks or neutralizes malodor. These may include (1) antimicrobial or bactericidal agents that kill the bacteria responsible for malodor production, (2) agents that inhibit or suppress or interfere with the bacterial enzymatic pathway that produces malodor, and (3) agents that mask or absorb or neutralize malodor. “Fragrances” as used herein are not considered deodorant active ingredients. Examples of deodorant actives ingredients include triclosan, triclocarban, usnic acid salts, zinc phenolsulfonate, b-chloro-D-alanine, D-cycloserine, animooxyacetic acid, cyclodextrine, and sodium bicarbonate. Alternatively, or in addition, the antiperspirant product 11 may comprise fragrances, for example, in an amount that imparts a long-lasting fragrance to the antiperspirant product 11.

Referring to FIG. 5, a personal care product 10 in accordance with another exemplary embodiment is provided. The personal care product 10 comprises an antiperspirant product 11 having an anhydrous aerosol formulation, which is substantially free of water, stored in a container 62. The aerosol formulation comprises the cooling sensation agent bound to silica as discussed in the foregoing paragraphs except preferably without the incorporation of water and/or alcohol, and includes other various ingredients, such as, for example, an active antiperspirant salt, talc, one or more fragrances, and a gaseous propellant, such as, one or more types of light hydrocarbons and/or fluorocarbons. The antiperspirant product 11 is contained in the container 62 under pressure from the gaseous propellant. When the plunger cap 64 is depressed, the valve stem 66 is actuated against a spring 68 to an open position allowing the liquid antiperspirant product 11 to be forced, via a gaseous propellant, through a tube 65, the valve stem 66 and finally out of a small hole in the plunger cap 64 as an aerosol or mist 70. Other suitable aerosol valve-container arrangements known to those skilled in the art may also be used with the various embodiments of the present invention.

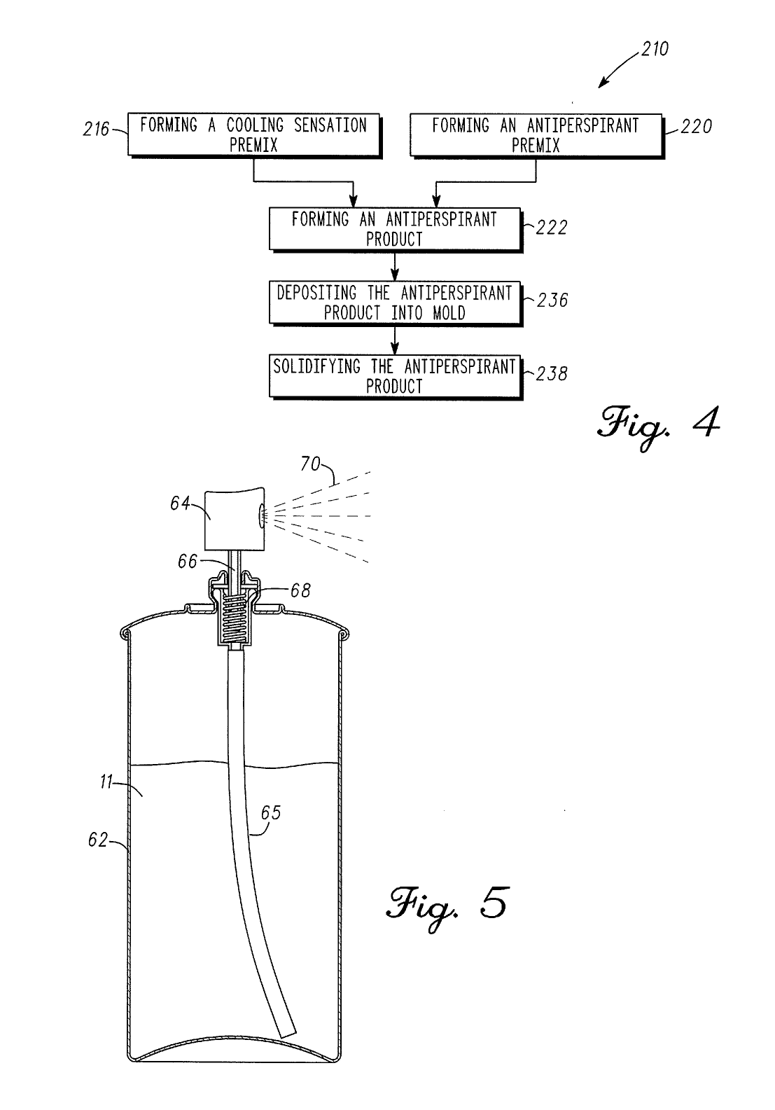

In accordance with exemplary embodiments, a method for manufacturing the antiperspirant product illustrated in FIGS. 1 and 2 is shown in FIGS. 3-4. With reference to FIG. 4, the method generally denoted at 210 comprises mixing a cooling sensation agent and silica particles and forming a cooling sensation agent premix (step 216). In one example, the cooling sensation agent is pre-blended with water and special denatured (SD) alcohol prior to being mixed with the silica particles. The cooling sensation agent blend preferably comprises by weight from about 50 to about 80% cooling sensation agent, from about 5 to about 15% water and from about 10 to about 25% SD alcohol. The cooling sensation agent blend is then mixed with the silica particles (e.g. porous hydrophilic silica particles) preferably using a low shear mixing process until the cooling sensation agent blend has been absorbed homogeneously (e.g. adsorbed onto surfaces and absorbed into the pores) into the silica particles. A hold time may be used after mixing to allow for a more homogeneous blend of the premix while preventing over-mixing, which could lead to some breakdown of the silica particles. In one embodiment, the cooling sensation agent premix comprises by weight from about 50 to about 80%, preferably from about 60 to about 75%, of the cooling sensation agent blend and from about 25 to about 40% of the silica particles, producing the cooling sensation agent premix comprising from about 3 to about 10 wt. % of water and from about 6.5 to about 16 wt. % of SD alcohol. Without being limited by theory, it is believed that the water and SD alcohol used in the cooling sensation agent blend facilitates adsorption of the cooling sensation agent onto surfaces and absorption into pores of hydrophilic silica particles during mixing. Mixers that work well are plow shear mixers, conical blade mixers or by hand for lab scale batches. Other suitable mixers known to those skilled in the art may be used.

An active antiperspirant compound is mixed with a structurant to form an antiperspirant premix (step 220). Other ingredients including hot melt waxes, silicones, polyhydric alcohols, paraffinic hydrocarbons, glycols, etc. also can be added during or after formation of the antiperspirant premix. The cooling sensation agent premix is then combined and mixed with the antiperspirant premix (step 222) in suitable proportions to form the antiperspirant product. In one embodiment, the antiperspirant product comprises the cooling sensation agent in an amount from about 0.2 to about 2.5 wt. %, silica particles in an amount of from about 0.5 to about 4 wt. %, SD alcohol in an amount of from about 0.1 to about 1 wt. %, and water in an amount of from about 0.1 to about 1 wt. %. The mixing disperses the silica particles, which have the cooling sensation agent captured thereon, throughout the antiperspirant product.

Referring to FIGS. 3-4, the antiperspirant product is molded directly into a mold by depositing the antiperspirant product in molten form into the mold (step 236) and solidifying the antiperspirant product (step 238). The molten temperature of the antiperspirant product is generally in the range of from about 65 to about 85° C., and the antiperspirant product is cooled below about 40° C., to produce the antiperspirant product in a non-molten form. The container 12 may be used as the mold for the antiperspirant product to form the antiperspirant product 11 illustrated in FIG. 3. It will be appreciated, however, that the invention is not limited to use of the container as a mold and that any satisfactory mold may be used for manufacturing the antiperspirant product.

The container 12 has an application end 24 and an opposite end 26. The container 12 also contains a factory seal 28, which is positioned over the application surface 14 of antiperspirant product 11 to protect it during shipment and to render it tamper-proof prior to purchase, and a cover 30. The factory seal 28 is removed by the user, and the cover is used during storage of the product between uses. As the product is exhausted, it is advanced from the container 12 by the user using advancement device 32, e.g., a screw mechanism as shown, at opposite end 26 of container 12.

As discussed in U.S. Pat. No. 5,753,212 filed Sep. 16, 1996, which is incorporated herein by reference in its entirety, the antiperspirant product 11 may be molded into the container 12 by first sealing the application end 24 of the container 12 with the factory seal 28 and introducing a predetermined quantity of molten antiperspirant product 11 through the open opposite end 26. The antiperspirant product 11 is then cooled to its non-molten form for example, by passing the filled container through a forced air tunnel operating at between about 10 to about 25° C. The finished product (FIG. 3) is completed by sealing the open opposite end 26 with a package base (not shown) that includes the advancement device 32. Other suitable methods for molding and/or forming the antiperspirant product known to those skilled in the art may also be used.

The following are examples of the antiperspirant product in accordance with the present invention. The examples are provided for illustration purposes only and are not meant to limit the various embodiments of the antiperspirant product in any way. All materials are set forth in weight percent.

### EXAMPLES

### Antiperspirant Product Composition

Antiperspirant Product—Solid Wax Formulation

| Ingredient | Wt. % |
|---|---|
| Cyclopentasiloxane | 30 to 40 |
| Stearyl Alcohol | 15 to 24 |
| Aluminum Zironium | 16 to 25 |
| Pentachlorohydrex | |
| PPG-14 Butyl Ether | 7 to 11 |
| Hydrogenated Castor Oil | 2 to 6 |
| Myristal Myristate | 1 to 4 |
| Silica (Aerosil 300) | 0.1 to 1 |
| Silica Dimethyl Silylate | 0.5 to 4 |
| Takasago Hot Act | 0.01 to 0.5 |
| Menthol | 0.2 to 2.5 |
| Water | 0.1 to 1 |
| SD Alcohol | 0.10 to 1 |
| Sipernat ® 22S | 0.50 to 4 |
| Fragrance | 0 to 4 |

Antiperspirant Product—Anhydrous Aerosol Formulation

| Ingredient | Wt. % |
|---|---|
| Butane | 35 to 45 |
| Cyclomethicone | 20 to 30 |
| Hydrofluorocarbon | 10 to 30 |
| Aluminum Chlorohydrate | 8 to 25 |
| Talc | 0.1 to 4 |
| Silica Dimethyl Silylate | 0.1 to 4 |
| Fragrance | 0.1 to 4 |
| Silica | 0.1 to 4 |
| Winsense Extra 500 | 0.1 to 2 |

Accordingly, antiperspirant products that exhibit antiperspirant efficacy and that also provide a cooling sensation effect that is perceived by the user have been described. The various embodiments of the antiperspirant products comprise an active antiperspirant ingredient or ingredients and a cooling sensation agent that is adsorbed onto surfaces and/or absorbed into pores of a plurality of silica particles that are dispersed throughout the product. By capturing and/or carrying the cooling sensation agent on the silica particles, the cooling sensation agent does not readily evaporate including during manufacturing of the product, thereby minimizing evaporative loss of the cooling sensation agent from the antiperspirant composition. Preferably, the silica particles are porous and hydrophilic such that when the antiperspirant product is applied to an underarm and the user perspires, the perspiration is readily absorbed by the silica particles, displacing and/or driving out the cooling sensation agent. As such, the cooling sensation agent intermingles with the perspiration and is absorbed into the skin, producing a cooling sensation effect that is perceived by the user. Also, by capturing and/or carrying the cooling sensation agent on the silica particles that are effective to release the cooling sensation agent when contacted with perspiration, the antiperspirant product has an excellent shelf-life and provides the user a cooling sensation effect throughout the day when it is most needed, e.g., when the user perspires.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A personal care product comprising:
a container; and
an antiperspirant product housed within the container and comprising a dispersed cooling sensation agent that is adsorbed onto surfaces of silica particles that are dispersed throughout the antiperspirant product, absorbed into pores of the silica particles or a combination thereof.

2. The personal care product according to claim 1, wherein the dispersed cooling sensation agent comprises menthol, vanillyl butyl ether, peppermint oil, methane carboxamide ethyl pyridine, menthoxypropanediol, menthanediol, cyanomethylphenyl methane carboxamide, camphor, ethyl menthane carboxamide, menthyl diisopropyl propionamide, menthyl lactate, 4-(butoxymenthyl)-2-methoxy-phenol, 3-[[5-methyl-2-(1-methyl)cyclohexyl]oxy]-1,2-propanediol, isopulegol, or a mixture thereof.

3. The personal care product according to claim 1, wherein the antiperspirant product comprises the dispersed cooling sensation agent in an amount of from about 0.2 to about 2.5 wt. %.

4. The personal care product according to claim 1, wherein the silica particles are porous hydrophilic silica particles and the dispersed cooling sensation agent is adsorbed onto and absorbed into the porous hydrophilic silica particles.

5. The personal care product according to claim 1, wherein the silica particles are effective to release the dispersed cooling sensation agent when contacted with body generated moisture.

6. The personal care product according to claim 1, wherein the antiperspirant product comprises the silica particles in an amount of from about 0.5 to about 4 wt. %.

7. The personal care product according to claim 1, wherein the antiperspirant product comprises SD alcohol in an amount of from about 0.1 to about 1 wt. %.

8. The personal care product according to claim 1, wherein the antiperspirant product comprises water in an amount of from about 0.1 to about 1 wt. %.

9. The personal care product according to claim 1, wherein the silica particles have a BET surface area of from about 50 to about 1000 m²/g.

10. The personal care product according to claim 1, wherein the silica particles have particle sizes d₅₀ of from about 2 to about 130 μm.

11. The personal care product according to claim 1, wherein the silica particles have a dioctyl acetate (DOA) absorption of from about 150 to about 400 DBP/100 g silica.

12. The personal care product according to claim 1, wherein the antiperspirant product is a solid wax formulation.

13. The personal care product according to claim 1, wherein the antiperspirant product is an anhydrous aerosol formulation.

14. The personal care product according to claim 13, wherein the antiperspirant product comprises the dispersed cooling sensation agent in an amount of from about 0.1 to about 2 wt. %.

15. The personal care product according to claim 13, wherein the antiperspirant product comprises the silica particles in an amount of from about 0.1 to about 4 wt. %.

16. A method for manufacturing an antiperspirant product, the method comprising the steps of:
depositing the antiperspirant product into a mold, the antiperspirant product comprising a dispersed cooling sensation agent that is adsorbed onto surfaces of silica particles that are dispersed throughout the antiperspirant product, absorbed into pores of the silica particles or a combination thereof; and
allowing the antiperspirant product to solidify.

17. The method according to claim 16, further comprising the steps of:
mixing a cooling sensation agent and the silica particles together to form a cooling sensation agent premix;
mixing antiperspirant ingredients together to form an antiperspirant premix; and
mixing the cooling sensation agent premix and the antiperspirant premix together to form the antiperspirant product.

18. The method according to claim 16, wherein the step of mixing the cooling sensation agent premix and the antiperspirant premix together comprises forming the antiperspirant product comprising the dispersed cooling sensation agent in an amount of from about 0.2 to about 2.5 wt. % and the silica particles in an amount of from about 0.5 to about 4 wt. %.

19. The method according to claim 16, wherein the step of mixing the cooling sensation agent comprises mixing water with the cooling sensation agent and the silica particles to form the cooling sensation agent premix comprising from about 3.0 to about 10 wt. % of water.

20. The method according to claim 19, wherein the step of mixing the cooling sensation agent comprises blending the water with the cooling sensation agent prior to mixing the cooling sensation agent with the silica particles.

21. The method according to claim 16, wherein the step of mixing the cooling sensation agent comprises mixing SD alcohol with the cooling sensation agent and the silica particles to form the cooling sensation agent premix comprising from about 6.5 to about 16 wt. % of SD alcohol.

22. The method according to claim 21, wherein the step of mixing the cooling sensation agent comprises blending the SD alcohol with the cooling sensation agent prior to mixing the cooling sensation agent with the silica particles.

23. The method according to claim 16, wherein the step of mixing the cooling sensation agent comprises mixing the cooling sensation agent with the silica particles to form the cooling sensation agent premix comprising from about 50 to about 80 wt. % of cooling sensation agent.

24. The method according to claim 16, wherein the step of depositing the antiperspirant product comprises depositing the antiperspirant product comprising the dispersed cooling sensation agent selected from the group consisting of menthol, vanillyl butyl ether, peppermint oil, methane carboxamide ethyl pyridine, menthoxypropanediol, menthanediol, cyanomethylphenyl methane carboxamide, camphor, ethyl menthane carboxamide, menthyl diisopropyl propionamide, menthyl lactate, 4-(butoxymenthyl)-2-methoxy-phenol, 3-[[5-methyl-2-(1-methyl)cyclohexyl]oxy]-1,2-propanediol, isopulegol, and mixtures thereof, and wherein the step of depositing a second portion comprises depositing a second portion comprising the solubilizer selected from the group consisting of polyglycol, polyethylene glycol, polypropylene glycol, and mixtures thereof.
